(19) United States
(12) Patent Application Publication
(10) Pub. No.: US 2009/0297464 A1
(43) Pub. Date: Dec. 3, 2009

(54) Cosmetic haircare process comprising a step for applying a crosslinked polyrotaxane, haircare compositions comprising a crosslinked polyrotaxane and uses thereof

(76) Inventor: Gwenaëlle Jegou, Livry Gargan (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/921,738

(22) PCT Filed: Jun. 8, 2006
(86) PCT No.: PCT/FR2006/001296

§ 371 Date: Jul. 7, 2009

Related U.S. Application Data
(60) Provisional application No. 60/727,451, filed on Oct. 18, 2005.

(30) Foreign Application Priority Data
Jun. 10, 2005 (FR) 0505922

(51) Int. Cl. A61K 8/72

(52) U.S. Cl. 424/70.2; 424/70.11

(57) ABSTRACT

The present patent application relates to a cosmetic haircare process comprising the use of a least one crosslinked polyrotaxane on the hair, the use of at least one crosslinked polyrotaxane on the hair possibly corresponding to the application to the hair of a cosmetic composition comprising at least one crosslinked polyrotaxane, or to the application to the hair of a cosmetic composition comprising at least one noncrosslinked polyrotaxane and to the crosslinking of the polyrotaxane(s) on the hair. The patent application also relates to an aqueous or anhydrous cosmetic haircare composition comprising at least one crosslinked polyrotaxane and at least one cosmetic adjuvant, and also to the uses of this composition in haircare.

The present invention relates to the field of cosmetic haircare compositions comprising a crosslinked polyrotaxane polymer.

Polyrotaxanes form part of the chemical family of inclusion compounds, which comprise a first molecular entity that forms a cavity of limited size in which is housed a molecular entity of a second chemical species.

In the field of cosmetic haircare, products for giving the hair volume and long-lasting shape are the subject of continued research.

More particularly, in the field of rinse-out products such as shampoos and hair conditioners, products are sought that are capable of affording, in wet or dry medium, not only good disentangling but also a bulk and body effect on the hair, in particular for fine hair.

Standard shampoos contain cationic polymers and silicone gums that generally give the hair good cosmetic properties, especially in terms of disentangling in wet or dry medium.

Shampoo formulations containing an adhesive polymer and/or a conditioning polymer, which afford a styling effect, are also known, especially from patent application FR 2 833 831.

However, these compositions cannot give the hair a volume effect.

In the field of “leave-in” styling compositions, such as styling gels or sprays, it is known practice to give the hair a styling effect and a pleasant feel by means of standard gelling or nongelling rigid polymers, such as the products sold under the trade name Carbopol by the company Goodrich or cellulose derivatives of carboxymethylcellulose type, or resins based on vinyl acetate and crotonic acid. However, the shaping of the hair is not long-lasting.

It is also known practice to use the polymers of Expancel type to improve the volume of a hairstyle, but these polymers have the drawback of requiring a high swelling activation temperature, above 100° C., which makes them difficult to use.

Recently, products such as carbon nanotubes, cationic latex particles or mineral particles have been used to increase hair volume, but the effects of these products generally remain insufficient.

The use of compositions comprising noncrosslinked pseudopolyrotaxanes for applications in cosmetics has also been described, especially in Japanese patent applications JP 09216815 and JP 09315937. However, these products are not crosslinked and do not have sufficient swelling properties and sufficient mechanical properties.

The term “pseudopolyrotaxane” means a supramolecular structure comprising at least one linear molecule and at least two cyclic molecules threaded onto said linear molecule, the linear molecule and the cyclic molecules not being linked via covalent bonds, and as such the cyclic molecules can move freely along the linear molecule.

These cyclic molecules are not blocked, they are threaded around the linear molecule and are thus capable of “leaving the chain”. Compositions comprising pseudopolyrotaxanes do not make it possible to obtain sufficient gelling of the composition and do not afford sufficient volume or sufficient shaping to the hair.

Furthermore, the mechanical properties of the compositions obtained are not always optimal; in particular, their elasticity is often poor.

More generally, in all haircare applications: washing, care, protection, shaping and dyeing of the hair, it is sought to improve the volume effect and the cosmetic effect.

The problem posed by the inventors of the present patent application is that of obtaining a cosmetic haircare process that can give hair a volume and body effect and shaping of the hairstyle, also accompanied by a good cosmetic effect.

The Applicant has just discovered, surprisingly and advantageously, that this problem can be solved by applying at least one crosslinked polyrotaxane to the hair.

The cosmetic process according to the present patent application makes it possible to give the hair a volume and styling result that diminishes much less quickly over time than the result imparted by using a standard composition.

The cosmetic haircare process according to the present patent application also makes it possible to give the hair good cosmetic properties, especially in terms of softness, elasticity and disentangling.

Another advantage of this process is that it uses cosmetic haircare compositions that have organoleptic properties, especially a texture that is particularly suited to haircare uses and also a pleasant feel. These cosmetic compositions are particularly thick.

The crosslinked polyrotaxane included in the cosmetic haircare compositions according to the present patent application also has mechanical and rheological properties that are suited to a cosmetic use, especially a haircare use, such as good elasticity and high resistance to breaking.

A first subject of the present patent application consists of a cosmetic haircare process comprising the use of at least one crosslinked polyrotaxane on the hair, the use of at least one crosslinked polyrotaxane on the hair possibly corresponding to the application to the hair of a cosmetic composition comprising at least one crosslinked polyrotaxane, or alternatively to the application to the hair of a cosmetic composition comprising at least one noncrosslinked polyrotaxane and crosslinking of the polyrotaxane(s) on the hair.

According to a first variant, the polyrotaxane is introduced into the aqueous phase of a cosmetic composition comprising a main aqueous phase.

According to a second variant, the polyrotaxane is introduced into the anhydrous phase of a cosmetic composition that is preferably anhydrous.

According to a third variant, the polyrotaxane is introduced into the aqueous phase of a cosmetic composition comprising a main anhydrous phase.

For the purposes of the present patent application, the expression “main phase of a composition” means that this phase represents at least 50% by weight relative to the weight of the total composition.

A second subject of the present patent application consists of an aqueous or anhydrous cosmetic haircare composition, containing at least one crosslinked polyrotaxane and at least one cosmetic adjuvant chosen from surfactants, polymers, ceramides and pseudoceramides, vitamins and provitamins, sunscreens, pigments, nacres or opacifiers, direct dyes, dye precursors, sequestrants, plasticizers, solubilizers, acidifying agents, basifying agents, neutralizers, mineral and organic thickeners, antioxidants, hydroxy acids, solvents, penetrants, buffers, dispersants, conditioning agents and preserving agents.

A third subject of the present patent application consists of the use of a crosslinked polyrotaxane in cosmetic haircare.

Other subjects, characteristics, aspects and advantages of the present invention will emerge even more clearly on reading the description and the examples that follow.

For the purposes of the present patent application, the term “cosmetic haircare composition” means a cosmetic composition in particular for washing, caring for or shaping the hair.

The cosmetic compositions according to the present patent application are preferentially in the form of gels, i.e. a three-dimensional network of molecules that retains in its mesh a large amount of solvent. The formation of such a network constitutes its gelling; the gelling of the cosmetic compositions according to the present patent application is particularly uniform and stable.

A “polyrotaxane” is obtained from a pseudopolyrotaxane to which is attached, at each end of the linear molecule, a molecular structure that prevents the cyclic molecules and the linear molecule from becoming separated, where appropriate.

The term “crosslinked polyrotaxane” means a compound comprising at least a first and a second polyrotaxane, at least one cyclic molecule of the first polyrotaxane and at least one cyclic molecule of the second polyrotaxane being linked via at least one bond that may be chemical or physical. The bond may especially be a metallic bond, an ionic bond, a covalent bond, an interaction resulting from the formation of charge-transfer complexes, or a weak interaction of hydrogen bonding, Van der Waals bonding or π-π bonding type, or a mixture thereof.

A polyrotaxane is thus a supramolecular assembly in which cyclic molecules are “included” by a linear molecule. To prevent cyclic molecules from unthreading from the linear molecule, the ends of the linear molecule are functionalized with bulky or ionic groups.

According to the present invention, the crosslinking of the polymer may be performed before or after its application to the keratin fibers.

In the present invention, the term “linear molecule” is intended to denote a substantially “linear” molecule. This means that a linear molecule may comprise one or more branched chains, provided that the cyclic molecules can be rotated about or move along the linear molecule.

The length of the “linear” molecule is not limited to any particular length, provided that the linear molecule allows the cyclic molecules to rotate about themselves or to move along said linear molecule.

The linear molecules used according to the present invention may be chosen from polymers, in particular:
hydrophilic polymers such as poly(vinyl alcohol), polyvinylpyrrolidone, poly((meth)acrylic acid), cellulose-based resins (carboxymethylcellulose, hydroxyethylcellulose, hydroxypropylcellulose and the like), polyacrylamide, polyethylene glycols, polypropylene glycols, polytetrahydrofurans, polyvinyl acetal-based resins, polyvinyl methyl ether, polyamines, polyethyleneimine, starch and the like, and/or copolymers thereof;
hydrophobic polymers such as polyolefinic resins such as polyethylene, polypropylene and resins of a copolymer with other olefinic monomers, polyester resins, polyisoprenes, polyisobutylenes, polybutadienes, polydimethylsiloxanes, polyethylenes and polypropylenes, poly(vinyl chloride) resins, polystyrene-based resins such as polystyrene and acrylonitrile-styrene copolymers, acrylic resins such as acrylonitrile-methyl acrylate copolymer resins, polycarbonate resins, polyurethane resins, vinyl chloride-vinyl acetate copolymer resins, polyvinyl butyral resins, ethylene/butylene copolymers; and derivatives thereof,
poly(methyl methacrylate) and (meth)acrylic ester copolymers, ethylenic (co)polymers comprising, for example, (meth)acrylic acid, (meth)acrylamide, vinyl, allylic or ethylene units, as dienes, or mixtures thereof.

Among these compounds, the ones that are preferred are polyethylene glycols, polyisoprene, polyisobutylene, polybutadienes, polypropylene glycols, polytetrahydrofurans, polydimethylsiloxanes, polyethylenes and polypropylenes. The ones that are particularly preferred are polyethylene glycols and polypropylene glycols.

The linear molecules advantageously have, independently of each other, a weight molecular mass of greater than or equal to 350 g/mol, for example ranging from 350 to 2 000 000, preferably ranging from 1500 to 1 000 000, more preferably from 2800 to 800 000, for example ranging from 7000 to 700 000 or from 10 000 to 600 000.

The linear molecules used according to the present invention preferably bear reactive groups at each end. The fact that they bear reactive groups facilitates the reaction with the molecular structures intended to prevent the separation between the linear molecules and the cyclic molecules that they bear.

The reactive groups depend on the blocking molecular structures to be used. Examples that may be mentioned include hydroxyl, amino and tosylate groups, polymerizable groups, activated esters such as N-hydroxysuccinimide esters, carboxyl and thiol, and the like.

In the present invention, a “cyclic molecule” denotes a molecule comprising at least one ring structure. The cyclic molecule may comprise two or more ring structures or double rings, or may be a macrocycle, for instance cyclodextrin.

Examples of cyclic molecules in the present invention may comprise:
cyclodextrins, for example alpha-cyclodextrin, beta-cyclodextrin, gamma-cyclodextrin, dimethylcyclodextrin and glucosylcyclodextrin, and derivatives thereof,
crown ethers,
benzo-crowns, dibenzo-crowns and dicyclohexano-crowns, and derivatives thereof.

The size of the internal cavity of the cyclic molecules may vary as a function of the chosen linear molecule.

In any event, cyclic molecules that may be threaded along the chain of the linear molecule are chosen. Thus, the cavity of the cyclic molecule will preferably have a diameter greater than the diameter of the right-vertical cross section of a minimum imaginary cylinder in which the linear molecule may be included.

When a cyclic molecule having a relatively large cavity and a cylindrical linear molecule having a relatively small diameter are used, several linear molecules may be included in the cavity of the cyclic molecule.

Among the cyclic molecules that may be used, cyclodextrins are preferred, and more particularly alpha-cyclodextrin.

According to one embodiment, alpha-cyclodextrin is used as cyclic molecule and polyethylene glycol is used as linear molecule.

The cyclic molecules preferably bear groups capable of giving rise to bonds that are not located in their cavity. This makes it possible to subsequently link the cyclic molecules together via chemical or physical bonding. The reactive groups of the cyclic molecules may comprise, for example, hydroxyl, amino, carboxyl or thiol groups. Furthermore, it is preferable to choose cyclic molecules bearing reactive groups that do not react with the structures blocking the chain ends during the blocking reaction between said blocking structures and the linear molecules.

The ratio between the number of cyclic molecules threaded onto the linear molecule and the maximum amount of cyclic molecules of the same nature that may be threaded onto this linear molecule ranges from 0.001 to 0.6, preferably from 0.01 to 0.5 and better still from 0.05 to 0.4. This ratio may be referred to as the “inclusion quantity”.

The maximum inclusion quantity is normalized as being equal to 1. It corresponds to the amount to which a linear molecule allows a maximum number of cyclic molecules to be included.

It is preferable for the linear molecule not to have a dense stacking of cyclic molecules. This dense stacking state corresponds to an inclusion quantity equal to 1. Creating a nondense stack of cyclic molecules makes it possible to conserve molecular segments that may be moved, and as such the crosslinked polyrotaxane has high resistance to breaking, high entropic elasticity, greater expandability and/or greater restorability, and, if so desired, high absorbing power or a highly hygroscopic nature.

According to another embodiment, the crosslinked polyrotaxane comprises cyclic molecules that each comprise at least two rings, in particular bicyclic molecules. In this embodiment, the linear molecule of the first polyrotaxane is threaded into the first ring of each bicyclic molecule and the linear molecule of the second polyrotaxane is threaded into the second ring of at least one bicyclic molecule. After mixing the linear molecules and the bicyclic molecules, each end of the linear molecules is blocked with a blocking group so as to prevent the removal of the bicyclic molecules in their skewered stated.

In this embodiment, it is not necessary to create bonds between the cyclic molecules by a crosslinking reaction, since the covalent bond connecting the two rings of the bicyclic molecule accordingly gives the polyrotaxane the crosslinked nature.

The bicyclic molecule may comprise, besides the two main rings, one or more other nuclei.

According to one embodiment, the cyclic molecules may be cyclized after inclusion of the linear molecules. More specifically, a precursor of the cyclic molecules having at least one open segment analogous to the letter “C” may be used.

In this case, the “C”-shaped segments may be closed after inclusion of the linear molecule, or after blocking of the linear molecule with a blocking group. For the molecules having a segment analogous to the letter “C”, see M. Asakawa, et al., edition Angewandte Chemie-International 37(3), 333-337 (1998), and M. Asakawa, et al., European Journal of Organic Chemistry 5, 985-994 (1999), both being incorporated herein by way of reference.

The blocking structures should hold the cyclic molecules threaded on the linear molecule.

These blocking structures may prevent the cyclic molecules from becoming separated from the linear molecule especially on account of their steric volume.

The blocking structures, for example located at each end of each linear molecule, may also prevent the cyclic molecules from becoming separated, for example from decomplexing from the linear molecule especially by bearing ionic charges.

The term “molecular structure” denotes herein a molecule, a macromolecule or a solid support, or a mixture.

A macromolecule or a solid support may contain several blocking sites. A blocking structure of a macromolecule may be present in the main chain or in a side chain.

When a blocking structure is a macromolecule A, the macromolecule A may constitute a matrix, part of which contains pseudopolyrotaxanes, or conversely the pseudopolyrotaxane may constitute a matrix, part of which contains the macromolecule A.

The molecular structures may be chosen from:
dinitrophenyl groups such as 2,4- and 3,5-dinitrophenyl groups;
cyclodextrins;
adamantane groups;
trityl groups;
fluoresceins;
pyrenes;
naphthalimides; and
combinations thereof.

According to one embodiment, when the linear molecule is a polyethylene glycol, the cyclic molecules may be chosen from alpha-cyclodextrin, dinitrophenyl groups such as 2,4- and 3,5-dinitrophenyl groups, adamantane groups, trityl groups, fluoresceins and pyrenes, and combinations thereof.

According to one variant, the crosslinked polyrotaxanes comprise at least a first and a second polyrotaxane, the linear molecule of the first polyrotaxane being threaded into the first ring of a bicyclic molecule and the linear molecule of the second polyrotaxane being threaded into the second ring of the bicyclic molecule.

According to another variant, the crosslinked polyrotaxanes comprise at least a first and a second polyrotaxane, at least one cyclic molecule of the first polyrotaxane and at least one cyclic molecule of the second polyrotaxane being linked via at least one chemical or physical bond.

When the bond is a chemical bond, the chemical bond may be formed by a single bond or by a bond involving various atoms or molecules. Said bond may be obtained by reaction of said two cyclic molecules with a cross-linking agent, a coupling agent, a photocrosslinking agent, under the action of temperature, of a variation in pH and/or of an irradiation.

A cyclic molecule preferably contains one or more reactive groups on the exterior of the nucleus, as described above. In particular, after the formation of a blocked polyrotaxane molecule, the cyclic molecules of different polyrotaxanes are preferably crosslinked together by means of a crosslinking agent. This reaction may also take place under the action of the temperature, a variation in pH or an irradiation. In this case, the conditions of the crosslinking reaction should be conditions under which the blocking groups of the blocked polyrotaxane are not removed.

Crosslinking agents that may be used are crosslinking agents that are well known in the prior art. Examples that may be mentioned include cyanuric chloride, trimesoyl chloride, terephthaloyl chloride, epichlorohydrin, dibromobenzene, glutaraldehyde, phenylene diisocyanates, tolylene diisocyanates (for example 2,4-tolylene diisocyanate), 1,1′-carbonyldiimidazole, divinyl sulfone, acid dichlorides (for example sebacoyl dichloride), acids substituted with a trichloro group, and the like. Various types of coupling agent may also be incorporated, such as coupling agents of silane type (for example various alkoxysilanes) and titanium-based coupling agents (for example various alkoxytitaniums). Other examples that may be mentioned include various photocrosslinking agents that are used for materials intended for soft contact lenses, for example photocrosslinking agents based on stilbazolium salts such as formylstyrylpyridium salts (see K. Ichimura, et al., Journal of Polymer science, polymer chemistry edition 20, 1411-1432 (1982) which is incorporated herein by way of reference), and other photocrosslinking agents, for example photodimerization-mediated photocrosslinking agents, specifically cinnamic acid, anthracene and thymines, and the like.

The crosslinking agents preferably have molecular masses of less than 2000 g/mol, preferably less than 1000, better still less than 600 and most particularly less than 400.

When alpha-cyclodextrin is used as cyclic molecule and when a crosslinking agent is used to crosslink it, examples of crosslinking agents that may be mentioned include cyanuric chloride, 2,4-tolylene diisocyanate, 1,1′-carbonyldiimidazole, trimesoyl chloride, terephthaloyl chloride and alkoxysilanes such as tetramethoxysilane and tetraethoxysilane, and the like. It is particularly preferable to use alpha-cyclodextrin as cyclic molecule and cyanuric chloride as crosslinking agent.

The compounds according to the present invention may be prepared according to the teaching of patent application EP 1 283 218.

First, the cyclic molecules and the linear molecules are mixed together to prepare the pseudopolyrotaxanes in which the cyclic molecules are threaded onto the linear molecules. Next, the polyrotaxanes are prepared by blocking each end of the linear molecules with blocking groups so as to prevent the removal of the cyclic molecules. Finally, two or more than two polyrotaxanes are crosslinked by linking the cyclic molecules via chemical bonds, so as to obtain the crosslinked polyrotaxane.

According to one embodiment of the invention, alpha-cyclodextrin is used as cyclic molecule, a polyethylene glycol is used as linear group, a 2,4-dinitrophenyl group is used as blocking group and cyanuric chloride is used as crosslinking agent.

First, each end of the polyethylene glycol is converted into an amino group, so as to be able subsequently to attach a blocking group to the end of the polyethylene glycol and to form the polyrotaxane. In one variant, diamine-terminated PEG/PPO copolymers sold by Hunstman under the reference Jeffamine may be used.

The alpha-cyclodextrin and the aminopolyethylene glycol derivative are then mixed together to prepare the pseudopolyrotaxane. The mixing time ranges from 1 to 48 hours and the mixing temperature ranges from 0 to 100° C., such that the inclusion quantity of the alpha-cyclodextrin on the polyethylene glycol derivative ranges from 0.001 to 0.6.

In general, a polyethylene glycol with an average molecular mass of 20 000 allows a maximum of 230 alpha-cyclodextrin molecules to be included. The maximum inclusion quantity corresponding to 230 molecules is equal to 1.

According to one embodiment, 60 to 65 (63) alpha-cyclodextrin molecules are on average threaded onto one polyethylene glycol molecule, which corresponds to a degree of inclusion ranging from 0.26 to 0.29 (0.28) relative to the maximum inclusion quantity. The inclusion quantity for alpha-cyclodextrin may be determined by NMR, light absorption or elemental analysis.

The pseudopolyrotaxane obtained is reacted with 2,4-dinitrofluorobenzene dissolved in DMF, which allows the polyrotaxane to be obtained.

The polyrotaxane is then dissolved in an aqueous sodium hydroxide solution, followed by addition of cyanuric chloride to crosslink the alpha-cyclodextrins.

The aqueous or anhydrous cosmetic haircare composition according to the present invention contains at least one crosslinked polyrotaxane and at least one cosmetic adjuvant chosen from surfactants, polymers, ceramides and pseudoceramides, vitamins and provitamins, sunscreens, pigments, nacres or opacifiers, direct dyes, dye precursors, sequestrants, plasticizers, solubilizers, acidifying agents, basifying agents, neutralizers, mineral and organic thickeners, antioxidants, hydroxy acids, solvents, penetrants, buffers, dispersants, conditioning agents and preserving agents.

Preferably, the cosmetic adjuvant is present in the composition in a content ranging from 0.1% to 20% and preferably from 1% to 10% by weight relative to the total weight of the cosmetic haircare composition.

Preferably, the crosslinked polyrotaxane is present in a content ranging from 0.01% to 80%, preferably from 3% to 30% and even more preferably from 5% to 25% by weight relative to the total weight of the composition. The cosmetic composition according to the present patent application may comprise one or more crosslinked polyrotaxanes.

The pH of the cosmetic compositions according to the invention is generally between 3 and 9 and preferably between 4 and 7.

The process according to the present patent application may be performed using an aqueous cosmetic haircare composition.

The aqueous cosmetic composition according to the present patent application contains a cosmetically acceptable medium comprising an aqueous phase.

For the purposes of the present patent application, the term “aqueous composition” means a composition containing from 50% to 100% by weight of water, preferably from 70% to 98% by weight of water and even more preferably from 85% to 95% by weight of water. Very preferably, the crosslinked polyrotaxane is in the aqueous phase of the aqueous cosmetic haircare composition.

The cosmetically acceptable medium used in the aqueous compositions according to the present patent application may also contain at least one alcohol and/or at least one additional organic solvent.

The alcohol used in the compositions according to the present invention is then a monohydroxylated alkanol chosen from C₁-C₄ lower alcohols, for instance ethanol, isopropanol, tert-butanol and n-butanol, ethanol being the alcohol preferably used.

The concentration of alcohol in the compositions according to the present invention is between 0 and 20%, preferably between 0.1% and 10% and even more preferably between 1% and 5% by weight relative to the total weight of the composition.

Preferably, the aqueous composition contains no C₁-C₄ alcohol.

Additional organic solvents which may be used in the aqueous compositions according to the present invention include polyols, for instance propylene glycol, and polyol ethers, and mixtures thereof.

The aqueous haircare compositions according to the present invention may be in any form that is suitable for application to the hair, preferably in the form of thickened lotions, aqueous or aqueous-alcoholic gels, creams or more or less hard pastes.

According to one particular embodiment of the invention, the cosmetic haircare process according to the present patent application is performed by carrying out the following steps:
application to the hair of an aqueous cosmetic composition comprising an aqueous phase containing a crosslinked polyrotaxane, said composition being, for example, a shampoo, a hair conditioner or a haircare lotion,
drying of said composition containing the cross-linked polyrotaxane, for example in ambient air or with a hair dryer, a hood or a curling iron,
water uptake of the dried composition containing the polyrotaxane polymer.

The step of water uptake of the dried composition may be performed by spraying an aqueous solution onto the keratin fibers, and it may also advantageously take place spontaneously on contact with the moisture of the ambient air.

The present patent application also relates to the haircare uses of an aqueous cosmetic composition comprising a crosslinked polyrotaxane.

According to a first variant, the present patent application relates to the use of the aqueous cosmetic composition comprising an aqueous phase containing a crosslinked polyrotaxane in hair washing products and haircare products, in particular in shampoos and hair conditioners.

The cosmetic compositions used according to the present patent application, in particular shampoos and hair conditioners, advantageously comprise silicones, preferably amino silicones, polymeric or nonpolymeric thickeners such as fatty alcohols and amides, and anionic, cationic, nonionic or amphoteric surfactants.

The present patent application also relates to a process for permanently reshaping the hair, in which an aqueous cosmetic composition comprising an aqueous phase containing a crosslinked polyrotaxane is applied to the hair.

According to the present application, the term “permanent reshaping” means the curling, permanent-waving or setting of Caucasian, Asiatic or North-African hair.

According to the present patent application, the term “relaxing” means the relaxing, straightening or defrizzing of Caucasian, Asiatic, North-African or African hair.

Processes for permanently reshaping or relaxing the hair generally use a reducing composition and then optionally a fixing composition, which is preferably oxidizing. In the permanent-reshaping or relaxing process according to the present patent application, the crosslinked polyrotaxane may be included either in the reducing composition, which then also comprises a reducing agent, preferably a sulfite, a bisulfite or a thiol, or in the oxidizing composition, which then also comprises an oxidizing agent, or in an additional composition intended to be applied before the reducing composition, after the reducing composition or after the oxidizing composition.

The present patent application also relates to a process for the temporary shaping of the hair, in which an aqueous cosmetic composition comprising an aqueous phase containing a crosslinked polyrotaxane is applied to the hair.

According to another variant, the present patent application relates to the uses of the aqueous cosmetic composition comprising an aqueous phase containing a crosslinked polyrotaxane for permanently reshaping or relaxing the hair.

According to another variant, the present patent application relates to the use of the aqueous cosmetic composition comprising an aqueous phase containing a crosslinked polyrotaxane in a composition for the direct dyeing or oxidation dyeing of the hair.

In the hair dyeing process according to the present patent application, the crosslinked polyrotaxane may be included in the dye composition, which also comprises at least one oxidation dye precursor chosen from oxidation bases and couplers and/or at least one direct dye. The crosslinked polyrotaxane may also be included in an additional composition intended to be applied before or after the dye composition.

According to another variant, the present patent application relates to the use of the aqueous cosmetic composition comprising an aqueous phase containing a crosslinked polyrotaxane in a hair gel, hair lotion or hair spray composition.

The uses of the aqueous cosmetic compositions make it possible to give keratin fibers additional volume and also good cosmetic properties in dry and wet medium (softness, disentangling) and good styling properties, especially in terms of curl bounce.

The process according to the present patent application may be performed using an anhydrous cosmetic haircare composition.

According to the present patent application, the term “anhydrous composition” means a composition containing less than 50% and down to 0% by weight of water, preferably between 1% and 20% by weight of water and even more preferably between 5% and 10% by weight of water.

According to one embodiment, the water optionally contained in the composition is not added to the ingredients during its preparation. This water may be in trace amount and may be due to the ingredients used to prepare said composition.

According to a first variant, the crosslinked polyrotaxane is in the anhydrous phase of the anhydrous cosmetic haircare composition.

According to a second variant, the crosslinked polyrotaxane is in the aqueous phase of the anhydrous cosmetic haircare composition.

Preferably, the crosslinked polyrotaxane is in the aqueous phase of the anhydrous cosmetic haircare composition.

The anhydrous haircare compositions according to the present invention may be in any form suitable for application to the hair, preferably in the form of an O/W or O/W/O lotion or alternatively in the form of W/O or W/O/W lotions.

The present patent application relates to the use of the anhydrous cosmetic composition comprising an anhydrous phase containing a crosslinked polyrotaxane in hair conditioning products.

This use makes it possible to give the hair good cosmetic properties in dry and wet medium, such as softness and disentangling. This use also makes it possible to give the hair volume. This use also makes it possible to improve the styling properties and in particular the curl bounce.

The anhydrous cosmetic haircare composition according to the present patent application advantageously comprises a fatty substance chosen from oils, waxes and pasty fatty substances.

The anhydrous cosmetic compositions in accordance with the present invention may comprise a fatty phase especially comprising oils, especially waxes, or fatty substances that are solid at room temperature (20-25° C.) and atmospheric pressure.

The term “oil” means any fatty substance that is in liquid form at room temperature (20-25° C.) and at atmospheric pressure. The liquid fatty phase may also contain, besides oils, other compounds dissolved in the oils, such as gelling agents and/or structuring agents.

The oil(s) may be present in a proportion of from 0.1% to 99% by weight, in particular from at least 1% to 90% by weight, more particularly from 5% to 70% by weight, especially from 10% to 60% by weight, or even from 20% to 50% by weight, relative to the total weight of the cosmetic composition according to the invention.

The additional and/or different oils of the copolymer according to the present invention that are suitable for preparing the cosmetic compositions according to the invention may be volatile or nonvolatile, silicone or nonsilicone oils.

For the purposes of the present invention, the term “volatile oil” means an oil (or nonaqueous medium) capable of evaporating on contact with the skin in less than one hour, at room temperature and at atmospheric pressure. The volatile oil is a volatile cosmetic oil, which is liquid at room temperature, especially having a nonzero vapor pressure, at room temperature and atmospheric pressure, in particular having a vapor pressure ranging from 0.13 Pa to 40 000 Pa (10⁻³ to 300 mmHg), preferably ranging from 1.3 Pa to 13 000 Pa (0.01 to 100 mmHg) and preferentially ranging from 1.3 Pa to 1300 Pa (0.01 to 10 mmHg).

For the purposes of the present invention, the term “nonvolatile oil” means an oil with a vapor pressure of less than 0.13 Pa. The volatile or nonvolatile oils may be hydrocarbon-based oils especially of animal or plant origin, synthetic oils, silicone oils or fluoro oils, or mixtures thereof.

For the purposes of the present invention, the term “silicone oil” means an oil comprising at least one silicon atom, and especially at least one Si—O group.

The term “hydrocarbon-based oil” means an oil mainly containing hydrogen and carbon atoms and possibly oxygen, nitrogen, sulfur and/or phosphorus atoms.

The volatile hydrocarbon-based oils may be chosen from hydrocarbon-based oils containing from 8 to 16 carbon atoms.

The fatty phase of the anhydrous cosmetic compositions according to the present invention may also comprise at least one volatile oil.

According to one particular embodiment, the anhydrous cosmetic compositions in accordance with the invention may also comprise at least one silicone oil chosen from volatile silicone oils and nonvolatile silicone oils, and mixtures thereof.

The nonvolatile oils may be present in the compositions according to the invention in a content ranging from 20% to 99% by weight, especially from 30% to 80% by weight and in particular from 40% to 80% by weight relative to the total weight of the composition.

Advantageously, the aqueous or anhydrous compositions according to the present invention contain at least one additional cosmetic additive chosen from thickening polymers, surfactants and conditioning agents.

According to a first preferred mode, the compositions according to the present invention contain at least one thickening polymer, also referred to as a “rheology modifier”.

The rheology modifiers may be chosen from fatty acid amides, cellulose-based thickeners, guar gum and derivatives thereof, gums of microbial origin, crosslinked homopolymers or copolymers of acrylic acid or of acrylamidopropanesulfonic acid and associative thickening polymers as described below.

These associative polymers are water-soluble polymers capable, in an aqueous medium, of reversibly combining with each other or with other molecules.

Their chemical structure comprises hydrophilic zones and hydrophobic zones characterized by at least one fatty chain.

The associative polymers may be of anionic, cationic, amphoteric or nonionic type.

Their concentration may range from about 0.01% to 10% and preferably 0.1% to 5% by weight relative to the total weight of the composition according to the invention.

Among the associative polymers of anionic type that may be mentioned are:
(I) those comprising at least one hydrophilic unit and at least one fatty-chain allyl ether unit,
(II) those comprising at least one hydrophilic unit of unsaturated olefinic carboxylic acid type, and at least one hydrophobic unit of unsaturated carboxylic acid (C₁₀-C₃₀)alkyl ester type,
(III) maleic anhydride/C₃₀-C₃₈ α-olefin/alkyl maleate terpolymers,
(IV) acrylic terpolymers comprising:
(a) about 20% to 70% by weight of an α,β-mono-ethylenically unsaturated carboxylic acid,
(b) about 20% to 80% by weight of a non-surfactant α,β-monoethylenically unsaturated monomer other than (a),
(c) about 0.5% to 60% by weight of a nonionic monourethane that is the product of reaction of a monohydric surfactant with a monoethylenically unsaturated monoisocyanate,
   such as those described in patent application EP-A-0 173 109,
   (V) copolymers comprising among their monomers an α,β-monoethylenically unsaturated carboxylic acid and an α,β-monoethylenically unsaturated carboxylic acid ester and an oxyalkylenated fatty alcohol.

Among the associative polymers of cationic type that may be mentioned are:
(I) the cationic associative polyurethanes whose family has been described by the Applicant in French patent application No. 0 009 609; it may be represented by the general formula (XVIII) below:

R-X-(P)ₙ[L-(Y)ₘ]ᵣ-L′-(P′)ₚ-X′-R′ (XVIII)
in which:
R and R′, which may be identical or different, represent a hydrophobic group or a hydrogen atom;
X and X′, which may be identical or different, represent a group comprising an amine function possibly bearing a hydrophobic group, or alternatively the group L″;
L, L′ and L″, which may be identical or different, represent a diisocyanate derivative;
P and P′, which may be identical or different, represent a group comprising an amine function possibly bearing a hydrophobic group;
Y represents a hydrophilic group;
r is an integer between 1 and 100, preferably between 1 and 50 and in particular between 1 and 25;
n, m and p are, independently of each other, between 0 and 1000;
the molecule containing at least one protonated or quaternized amine function and at least one hydrophobic group;
(II) quaternized cellulose derivatives and polyacrylates containing noncyclic amino side groups.

The amphoteric associative polymers are preferably chosen from those comprising at least one noncyclic cationic unit. Even more particularly, the preferred polymers are those prepared from or comprising 1 to 20 mol %, preferably 1.5 to 15 mol % and even more particularly 1.5 to 6 mol % of monomer comprising a fatty chain, relative to the total number of moles of monomers.

Advantageously, the amount of thickeners present in the composition according to the invention is between 0.01% and 10% and preferably between 0.1% and 5% by weight relative to the total weight of the composition.

According to a second preferred mode, the composition according to the present invention also contains at least one surfactant chosen from anionic, cationic, nonionic, amphoteric and zwitterionic surfactants.

Advantageously, the amount of surfactants present in the composition according to the invention is between 0.01% and 40% and preferably between 0.1% and 30% by weight relative to the total weight of the composition.

Conditioning agents that may be mentioned include silicones and cationic polymers.

The silicones that may be used in the compositions according to the present invention may be linear, cyclic, branched or unbranched, and volatile or nonvolatile. They may be in the form of oils, resins or gums, and may in particular be polyorganosiloxanes that are insoluble in the cosmetically acceptable medium.

For the purposes of the present invention, the expression “cationic polymer” denotes any polymer containing cationic groups and/or groups that may be ionized into cationic groups.

The preferred cationic polymers are chosen from those that contain units comprising primary, secondary, tertiary and/or quaternary amine groups, which may either form part of the main polymer chain, or be borne by a side substituent directly attached thereto.

Among the cationic polymers that may be mentioned more particularly are polymers of the polyamine, polyaminoamide and polyquaternary ammonium type.

Mention is made in particular of:
(1) The cellulose ether derivatives comprising quaternary ammonium groups, described in French patent 1 492 597, and in particular the polymers sold under the names JR (JR 400, JR 125, JR 30M) or LR (LR 400, LR 30M) by the company Union Carbide Corporation. These polymers are also defined in the CTFA dictionary as quaternary ammoniums of hydroxyethylcellulose that has reacted with an epoxide substituted with a trimethylammonium group.
(2) The cationic polysaccharides described more particularly in patents U.S. Pat. Nos. 3,589,578 and 4,031,307, such as guar gums containing trialkylammonium cationic groups. Guar gums modified with a salt (e.g. chloride) of 2,3-epoxypropyltrimethylammonium are used, for example.
Such products are sold especially under the trade names Jaguar C13 S, Jaguar C15, Jaguar C17 or Jaguar C162 by the company Meyhall.
(3) Cyclopolymers of alkyldiallylamine or of dialkyldiallylammonium. Mention may be made more particularly of the dimethyldiallylammonium chloride homopolymer sold under the name Merquat 100 by the company Calgon (and homologs thereof of low weight-average molecular mass) and the copolymers of diallyldimethylammonium chloride and of acrylamide sold under the name Merquat 550.

The cosmetic compositions according to the present patent application may also be used for repairing the hair.

A subject of the invention is also the use of the composition according to the invention in a formulation chosen from hair lotions, hair gels, hair mousses without propellent gases (“foamers”), hair creams, aerosol hair sprays with a propellent gas or hair sprays in a pump-dispenser bottle without propellent gas, for shaping or holding the hair.

The examples that follow illustrate the invention without limiting its scope.

### EXAMPLES

### Example 1

### Preparation of the Polyrotaxane

Polyethylene glycol biamine (0.9 g), abbreviated as PEG-BA, produced by Fluka, and α-cyclodextrin (3.6 g) were dissolved in water (30 mL) at 80° C. and the mixture was maintained at 5° C. overnight, to obtain the white paste of the inclusion complex. The paste was dried, an excess of 2,4-dinitrofluorobenzene (2.4 mL) was added at the same time as dimethylformamide (10 mL) and the mixture was stirred under a nitrogen atmosphere at room temperature overnight. The reaction medium was dissolved in DMSO (50 mL) and precipitated twice from aqueous 0.1% sodium chloride solution (800 mL) to give a yellow product. The product was collected, washed with water and methanol (three times, respectively) and dried to produce the polyrotaxane (1.25 g): the blocked polyrotaxane was thus obtained.

### Example 2

### Production of a Crosslinked Polyrotaxane

100 mg of polyrotaxane were dissolved in 0.5 mL of 1N sodium hydroxide solution at 5° C. in a reactor. 35 mg of 2,4,6-trifluoro-1,3,5-triazine dissolved in 1N NaOH (0.5 mL) were added to the reactor. The reaction mixture was reacted at room temperature for 3 hours to produce the crosslinked polyrotaxane.

In the examples that follow, the percentages are on a weight basis.

### Example 3

### Preparation of a Styling Hair Gel

2 g of an aqueous solution comprising 200 mg of the gel of example 2 and 0.4 mg of a preserving agent are applied to locks of 1.5 g of wet hair. The locks are massaged and then left to stand for 5 minutes.

A lock is wound round a curler 0.5 cm in diameter and heated under a hood at 70° C. for 30 minutes. The lock is then detached from the curler and left at ambient humidity. After drying, the tonicity of the curl, the feel of the hair and the disentangling of the locks with a comb are evaluated. Long-lasting curl bounce (>8 days) is observed, which is very much superior to that of the control lock treated only with an aqueous solution and a preserving agent.

### Example 4

### Preparation of a Styling Hair Gel

2 g of an aqueous solution comprising 200 mg of the gel of example 1 and 0.4 mg of a preserving agent is applied to locks of 1.5 g of wet hair pretreated with dilute 0.5N sodium hydroxide solution (2 mL) containing 20 mg of cyanuric chloride. A lock is wound round a curler 0.5 cm in diameter and dried under a hood at 70° C. for 1 hour. The lock is then detached from the curler and left at ambient humidity. After drying, the tonicity of the curl, the feel of the hair and the disentangling of the locks with a comb are evaluated. Long-lasting curl bounce (>8 days) is observed, which is very much superior to that of the control lock treated only with an aqueous solution and a preserving agent.

### Example 5

### Preparation of a Styling Hair Gel

2 g of an aqueous solution comprising 200 mg of the gel of example 2 and 0.4 mg of a preserving agent are applied to locks of 1.5 g of wet hair. The locks are massaged and then left to stand for 5 minutes.

A lock is wound round a curler 0.5 cm in diameter and heated under a hood at 70° C. for 30 minutes. The lock is then detached from the curler and left at ambient humidity. After drying, the tonicity of the curl, the feel of the hair and the disentangling of the locks with a comb are evaluated. Long-lasting curl bounce (>8 days) is observed, which is very much superior to that of the control lock treated only with an aqueous solution and a preserving agent.

### Example 6

### Preparation of a Volumizing Shampoo

2 g of an aqueous surfactant solution (12.5% AM of lauryl ether sulfate and 2.5% AM of cocoylbetaine) comprising 20 mg of the gel of example 2 above are applied to a 1.5 g wet lock. The lock is immersed in this solution for 3 minutes and then rinsed and dried. The lock is massaged, left to stand for 5 minutes and then rinsed. Before drying, the disentangling is evaluated. Very good disentangling in wet medium (after rinsing) is observed. After drying, the tonicity of the curl, the feel of the hair and the disentangling of the locks with a comb and in dry medium are evaluated. A lock volume very much greater than that of the control lock treated only with an aqueous solution comprising 12.5% AM of lauryl ether sulfate and 2.5% AM of cocoylbetaine is observed. Long-lasting curl bounce (>8 days) is observed, which is very much superior to that of the control lock treated only with an aqueous solution and a preserving agent.

### Example 7

### Preparation of a Volumizing Hair Conditioner

| Constituent | by weight |
|---|---|
| Behentrimonium chloride Genamin KDMP (Clariant) | 1.2 |
| PEG/PPG 22/24 dimethicone Mirasil DMCO (Rhodia) | 0.5 |
| Cyclopentasiloxane DC 245 fluid (Dow Corning) | 10.0 |
| Propylene glycol | 2.5 |
| Preserving agent | qs |
| Fragrance | qs |
| Citric acid/sodium hydroxide | qs pH 6.5 |
| Water | qs 100 |

20 mg of the above gel (example 2) are added to 2 g of the emulsion given in the above table. The composition obtained according to the present patent application is applied to a 1.5 g wet lock. The lock is massaged, left to stand for 5 minutes and then rinsed and dried. Very good disentangling is observed in wet medium (after rinsing), and a lock volume very much greater than that of the control lock treated only with the formulation after shampooing without crosslinked polyrotaxane is observed in dry medium.

Long-lasting curl bounce (>8 days) is observed, which is very much superior to that of the control lock treated only with an aqueous solution and a preserving agent.

### Example 8

### Preparation of a Permanent-Waving Composition

The following reducing composition is prepared:

| | |
|---|---|
| Crosslinked polyrotaxane according to example 2 | 3% |
| Thioglycolic acid | 8% |
| Monoethanolamine | qs pH 7 |
| Water | qs 100 |

This composition is applied to the hair and is then left to stand on the hair for half an hour. After rinsing, followed by a leave-on time of 10 minutes, a hydrogen peroxide oxidizing composition is applied and the hair is left to stand for a further half an hour. After rinsing and drying, it is found that the head of hair obtained has good cosmetic properties, especially in terms of softness.

### Example 9

### Preparation of a Hair Dye Composition

The dye composition, in accordance with the invention, below was prepared:

| | | |
|---|---|---|
| Crosslinked polyrotaxane according to example 2 | 2 | g |
| Oleic acid | 3 | g |
| Aqueous sodium bisulfite solution containing 35% | 0.45 | g AM* |
| AM* | | |
| para-Phenylenediamine | 0.162 | g |
| Resorcinol | 0.165 | g |
| Aqueous ammonia (20% NH₃) | 11.5 | g |
| Water | qs 100 | g |

*AM: = Active Material

At the time of use, this composition was mixed weight for weight with a 20-volumes aqueous hydrogen peroxide solution, and the mixture obtained was then applied to locks of natural hair containing 90% white hairs. After a leave-on time of 10 minutes, the locks were rinsed and then washed with a shampoo, rinsed again and then dried.

It is found that the head of hair obtained has good cosmetic properties, especially in terms of softness.

1. A cosmetic haircare process comprising treating the hair with at least one crosslinked polyrotaxane.

2. The process as claimed in claim 1, wherein a cosmetic composition comprising at least one crosslinked polyrotaxane is applied to the hair.

3. The cosmetic haircare process as claimed in claim 1, wherein a cosmetic composition comprising at least one noncrosslinked polyrotaxane is applied to the hair and crosslinking of the at least one noncrosslinked polyrotaxane is brought about on the hair.

4. The process as claimed in claim 1, wherein the at least one crosslinked polyrotaxane comprises at least a first polyrotaxane and at least a second polyrotaxane, wherein the first polyrotaxane and the second polyrotaxane independently comprise at least one linear molecule and at least two cyclic molecules.

5. The process as claimed in claim 4, wherein the at least one linear molecule of the first polyrotaxane and/or the at least one linear molecule of the second polyrotaxane are independently chosen from polyethylene glycols and polypropylene glycols.

6. The process as claimed in claim 4, wherein the at least one linear molecule of the first polyrotaxane and/or the at least one linear molecule of the second polyrotaxane are chosen, independently, from hydrophobic polymers.

7. The process as claimed in claim 1, wherein the at least one crosslinked polyrotaxane comprises at least a first polyrotaxane and at least a second polyrotaxane, wherein the first polyrotaxane and second polyrotaxane independently comprise at least one linear molecule and at least two cyclic molecules, wherein the at least one linear molecule of the first polyrotaxane and/or the at least one linear molecule of the second polyrotaxane are chosen, independently, from poly(methyl methacrylate) and (meth)acrylic ester copolymers, ethylenic polymers, ethylenic copolymers, and mixtures thereof.

8. The process as claimed in claim 4, wherein the at least one linear molecule of the first polyrotaxane and/or the at least one linear molecule of the second polyrotaxane have, independently of each other, a weight molecular mass of greater than or equal to 350 g/mol.

9. The process as claimed in claim 4, wherein the at least one linear molecule of the first polyrotaxane and/or the at least one linear molecule of the second polyrotaxane bear, independently of each other, reactive groups chosen from hydroxyl, amino, and tosylate groups, polymerizable groups, activated esters, carboxyl and thiol.

10. The process as claimed in claim 4, wherein, the at least two cyclic molecules comprise at least two rings, or the at least two cyclic molecules may be cyclized after the inclusion of the at least one linear molecules.

11. The process as claimed in claim 4, wherein the at least two cyclic molecules of the first and second polyrotaxanes are chosen from cyclodextrins, and crown ethers, benzo-crowns, dibenzo-crowns and dicyclohexano-crowns, and derivatives thereof.

12. The process as claimed claim 11, wherein the at least two cyclic molecules are chosen from alpha-cyclodextrins.

13. The process as claimed in claim 4, wherein for the first polyrotaxane and the second polyrotaxane independently the ratio between the number of the at least two cyclic molecules threaded onto the at least one linear molecule and the maximum amount of cyclic molecules of the same nature that may be threaded onto a linear molecule of the same nature ranges from 0.001 to 0.6.

14. The process as claimed in claim 4, wherein the at least one linear molecule of the first polyrotaxane and/or the at least one linear molecule of the second polyrotaxane comprise, at each end of the at least one linear molecule, independently, a molecular structure that prevents the at least two cyclic molecules and the at least one linear molecule from becoming separated, wherein the molecular structure is a molecule or a macromolecule.

15. The process as claimed claim 14, wherein the molecular structure bears an ionic charge and/or occupies a steric volume such that the molecular structure prevents the at least two cyclic molecules and the at least one linear molecule from becoming separated.

16. The process as claimed in claim 14, wherein the molecular structure is chosen from dinitrophenyl groups, cyclodextrins, adamantane groups, trityl groups, fluoresceins, pyrenes, naphthalimides, and combinations thereof.

17. The process as claimed in claim 4, wherein at least one cyclic molecule of the first polyrotaxane and at least one cyclic molecule of the second polyrotaxane are linked via a chemical bond, wherein said bond is obtained by reaction of the at least one cyclic molecule of the first polyrotaxane and the at least one cyclic molecule of the second polyrotaxane.

18. The process as claimed in claim 43, wherein the crosslinking agent is chosen from cyanuric chloride, trimesoyl chloride, terephthaloyl chloride, epichlorohydrin, dibromobenzene, glutaraldehyde, phenylene diisocyanates, tolylene diisocyanates, 1,1′-carbonyldiimidazole, divinyl sulfone, acid dichlorides, for example sebacoyl dichloride, and acids substituted with a trichloro group.

19. The process as claimed in claim 18, wherein the crosslinking agent is chosen from cyanuric chloride, 2,4-tolylene diisocyanate, 1,1′-carbonyldiimidazole, trimesoyl chloride and terephthaloyl chloride.

20. The process as claimed in claim 43, wherein the coupling agent is chosen from silane coupling agents and titanium-based coupling agents.

21. The process as claimed in claim 43, wherein the photocrosslinking agent is chosen from photocrosslinking agents based on stilbazolium salts, cinnamic acid, anthracene and thymines.

22. The process as claimed in claim 1, wherein the at least one crosslinked polyrotaxane is included in the aqueous phase of a cosmetic composition comprising a main aqueous phase.

23. The process as claimed in claim 1, comprising:
application to the hair of an aqueous cosmetic composition comprising an aqueous phase comprising the at least one crosslinked polyrotaxane,
drying of said composition comprising the at least one crosslinked polyrotaxane,
water uptake of the dried composition comprising the at least one polyrotaxane polymer.

24. The process as claimed in claim 23, wherein the step of water uptake of the dried composition is performed either by spraying an aqueous solution onto the hair, or by contact with the moisture of the ambient air.

25. The process as claimed in claim 1, wherein an aqueous cosmetic composition comprising an aqueous phase comprising the at least one crosslinked polyrotaxane is applied to the hair for temporarily reshaping the hair.

26. The process as claimed in claim 1, wherein an aqueous cosmetic composition comprising an aqueous phase comprising the at least one crosslinked polyrotaxane, a reducing composition and optionally a fixing composition are applied to the hair for permanently reshaping the hair.

27. The process as claimed in claim 26, wherein 1) the aqueous composition comprising the at least one crosslinked polyrotaxane is included in the reducing composition, wherein the reducing composition also comprises a reducing agent; 2) the aqueous composition is included in the oxidizing composition, wherein the oxidizing composition also comprises an oxidizing agent; or 3) the aqueous composition is an additional composition intended to be applied before the reducing composition, after the reducing composition or after the oxidizing composition is applied to the hair.

28. The process as claimed in claim 1, wherein an aqueous cosmetic composition comprising an aqueous phase comprising the at least one crosslinked polyrotaxane and a reducing composition and optionally a fixing composition are applied to the hair for relaxing the hair.

29. The process as claimed in claim 28, wherein 1) the aqueous composition comprising the at least one crosslinked polyrotaxane is included in the reducing composition, wherein the reducing composition also comprises a reducing agent; or 2) the aqueous composition is an additional composition intended to be applied before the reducing composition or after the reducing composition is applied to the hair.

30. The process as claimed in claim 1, wherein an aqueous cosmetic composition comprising an aqueous phase comprising the at least one crosslinked polyrotaxane, and a dye composition, are applied to the hair for direct dyeing or oxidation dyeing of the hair.

31. The process as claimed in claim 30, wherein 1) the aqueous composition comprising the at least one crosslinked polyrotaxane is included in the dye composition, wherein the dye composition also comprises at least one oxidation dye precursor chosen from oxidation bases and couplers and/or at least one direct dye; or 2) the aqueous composition is an additional composition intended to be applied before the dye composition or after the dye composition is applied to the hair.

32. The process as claimed in claim 1, wherein the at least one crosslinked polyrotaxane is included in the aqueous phase of a cosmetic composition comprising a main anhydrous phase.

33. The process as claimed in claim 32, wherein cosmetic composition comprising an anhydrous phase containing the at least one crosslinked polyrotaxane and an oil is applied to the hair for conditioning the hair.

34. An aqueous or anhydrous cosmetic haircare composition, comprising at least one crosslinked polyrotaxane as defined in claim 4, and at least one cosmetic adjuvant chosen from surfactants, polymers, ceramides, pseudoceramides, vitamins, provitamins, sunscreens, pigments, nacres, opacifiers, direct dyes, dye precursors, sequestrants, plasticizers, solubilizers, acidifying agents, basifying agents, neutralizers, mineral thickeners, organic thickeners, antioxidants, hydroxy acids, solvents, penetrants, buffers, dispersants, conditioning agents and preserving agents.

35. The cosmetic haircare composition as claimed in claim 34, wherein the cosmetic adjuvant is present in a content ranging from 0.1% to 20% by weight relative to the total weight of the cosmetic haircare composition.

36. The cosmetic haircare composition as claimed in claim 34, wherein the crosslinked polyrotaxane is present in a content ranging from 0.01% to 80% by weight relative to the total weight of the composition.

37. A hair washing or haircare product comprising the aqueous composition as claimed in claim 34.

38. A cosmetic haircare product comprising the aqueous composition as claimed in claim 34, for curling or relaxing the hair.

39. A cosmetic haircare product comprising the aqueous composition as claimed in claim 34, for dyeing the hair.

40. A leave-in hair conditioning product comprising the anhydrous composition as claimed in claim 34, wherein the anhydrous composition is in the form of an O/W, O/W/O, W/O or W/O/W lotion.

41. A hair gel product or hair lotion product comprising the aqueous composition as claimed in claim 34.

42. The process as claimed in claim 4, wherein the at least one linear molecule of the first polyrotaxane and/or the at least one linear molecule of the second polyrotaxane are independently chosen from hydrophilic polymers.

43. The process as claimed in claim 17, wherein the chemical bond is obtained with a crosslinking agent, a coupling agent, with a photocrosslinking agent, by variation in temperature, by variation in pH, and/or by irradiation.
